SEQUENCE LISTING (23 sequences)

SEQ ID NO: 1 - protein, 12 aa, artificial
  Consensus sequence from human CDK (Cyclin dependent kinases)
VVTXWYRXPXIL

SEQ ID NO: 2 - protein, 297 aa, homo sapiens
MEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 3 - protein, 298 aa, homo sapiens
MENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 4 - protein, 305 aa, homo sapiens
MDMFQKVEKIGEGTYGVVYKAKNRETGQLVALKKIRLDLEMEGVPSTAIREISLLKELKHPNIVRLLDVVHNERKLYLVFEFLSQDLKKYMDSTPGSELPLHLIKSYLFQLLQGVSFCHSHRVIHRDLKPQNLLINELGAIKLADFGLARAFGVPLRTYTHEVVTLWYRAPEILLGSKFYTTAVDIWSIGCIFAEMVTRKALFPGDSEIDQLFRIFRMLGTPSEDTWPGVTQLPDYKGSFPKWTRKGLEEIVPNLEPEGRDLLMQLLQYDPSQRITAKTALAHPYFSSPEPSPAARQYVLQRFRH

SEQ ID NO: 5 - protein, 303 aa, homo sapiens
MATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 6 - protein, 292 aa, homo sapiens [sequence omitted]

SEQ ID NO: 7 - protein, 326 aa, homo sapiens
MEKDGLCRADQQYECVAEIGEGAYGKVFKARDLKNGGRFVALKRVRVQTGEEGMPLSTIREVAVLRHLETFEHPNVVRLFDVCTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQSSYATPVDLWSVGCIFAEMFRRKPLFRGSSDVDQLGKILDVIGLPGEEDWPRDVALPRQAFHSKSAQPIEKFVTDIDELGKDLLLKCLTFNPAKRISAYSALSHPYFQDLERCKENLDSHLPPSQNTSELNTA

SEQ ID NO: 8 - protein, 346 aa, homo sapiens
MALDVKSRAKRYEKLDFLGEGQFATVYKARDKNTNQIVAIKKIKLGHRSEAKDGINRTALREIKLLQELSHPNIIGLLDAFGHKSNISLVFDFMETDLEVIIKDNSLVLTPSHIKAYMLMTLQGLEYLHQHWILHRDLKPNNLLLDENGVLKLADFGLAKSFGSPNRAYTHQVVTRWYRAPELLFGARMYGVGVDMWAVGCILAELLLRVPFLPGDSDLDQLTRIFETLGTPTEEQWPDMCSLPDYVTFKSFPGIPLHHIFSAAGDDLLDLIQGLFLFNPCARITATQALKMKYFSNRPGPTPGCQLPRPNCPVETLKEQSNPALAIKRKRTEALEQGGLPKKLIF

SEQ ID NO: 9 - protein, 464 aa, homo sapiens
MDYDFKVKLSSERERVEDLFEYEGCKVGRGTYGHVYKAKRKDGKDDKDYALKQIEGTGISMSACREIALLRELKHPNVISLQKVFLSHADRKVWLLFDYAEHDLWHIIKFHRASKANKKPVQLPRGMVKSLLYQILDGIHYLHANWVLHRDLKPANILVMGEGPERGRVKIADMGFARLFNSPLKPLADLDPVVVTFWYRAPELLLGARHYTKAIDIWAIGCIFAELLTSEPIFHCRQEDIKTSNPYHHDQLDRIFNVMGFPADKDWEDIKKMPEHSTLMKDFRRNTYTNCSLIKYMEKHKVKPDSKAFHLLQKLLTMDPIKRITSEQAMQDPYFLEDPLPTSDVFAGCQIPYPKREFLTEEEPDDKGDKKNQQQQQGNNHTNGTGHPGNQDSSHTQGPPLKKVRVVPPTTTSGGLIMTSDYQRSNPHAAYPNPGPSTSQPQSSMGYSATSQQPPQYSHQTHRY

SEQ ID NO: 10 - protein, 372 aa, homo sapiens
MAKQYDSVECPFCDEVSKYEKLAKIGQGTFGEVFKARHRKTGQKVALKKVLMENEKEGFPITALREIKILQLLKHENVVNLIEICRTKASPYNRCKGSIYLVFDFCEHDLAGLLSNVLVKFTLSEIKRVMQMLLNGLYYIHRNKILHRDMKAANVLITRDGVLKLADFGLARAFSLAKNSQPNRYTNRVVTLWYRPPELLLGERDYGPPIDLWGAGCIMAEMWTRSPIMQGNTEQHQLALISQLCGSITPEVWPNVDNYELYEKLELVKGQKRKVKDRLKAYVRDPYALDLIDKLLVLDPAQRIDSDDALNHDFFWSDPMPSDLKGMLSTHLTSMFEYLAPPRRKGSQITQQSTNQSRNPATTNQTEFERVF

SEQ ID NO: 11 - protein, 360 aa, homo sapiens
MAEPDLECEQIRLKCIRKEGFFTVPPEHRLGRCRSVKEFEKLNRIGEGTYGIVYRARDTQTDEIVALKKVRMDKEKDGIPISSLREITLLLRLRHPNIVELKEVVVGNHLESIFLVMGYCEQDLASLLENMPTPFSEAQVKCIVLQVLRGLQYLHRNFIIHRDLKVSNLLMTDKGCVKTADFGLARAYGVPVKPMTPKVVTLWYRAPELLLGTTTQTTSIDMWAVGCILAELLAHRPLLPGTSEIHQIDLIVQLLGTPSENIWPGFSKLPLVGQYSLRKQPYNNLKHKFPWLSEAGLRLLHFLFMYDPKKRATAGDCLESSYFKEKPLPCEPELMPTFPHHRNKRAAPATSEGQSKRCKP

SEQ ID NO: 12 - protein, 298 aa, Saccharomyces cerevisiae
MSGELANYKRLEKVGEGTYGVVYKALDLRPGQGQRVVALKKIRLESEDEGVPSTAIREISLLKELKDDNIVRLYDIVHSDAHKLYLVFEFLDLDLKRYMEGIPKDQPLGADIVKKFMMQLCKGIAYCHSHRILHRDLKPQNLLINKDGNLKLGDFGLARAFGVPLRAYTHEIVTLWYRAPEVLLGGKQYSTGVDTWSIGCIFAEMCNRKPIFSGDSEIDQIFKIFRVLGTPNEAIWPDIVYLPDFKPSFPQWRRKDLSQVVPSLDPRGIDLLDKLLAYDPINRISARRAAIHPYFQES

SEQ ID NO: 13 - protein, 297 aa, Schizosaccharomyces pombae
MENYQKVEKIGEGTYGVVYKARHKLSGRIVAMKKIRLEDESEGVPSTAIREISLLKEVNDENNRSNCVRLLDILHAESKLYLVFEFLDMDLKKYMDRISETGATSLDPRLVQKFTYQLVNGVNFCHSRRIIHRDLKPQNLLIDKEGNLKLADFGLARSFGVPLRNYTHEIVTLWYRAPEVLLGSRHYSTGVDIWSVGCIFAEMIRRSPLFPGDSEIDEIFKIFQVLGTPNEEVWPGVTLLQDYKSTFPRWKRMDLHKVVPNGEEDAIELLSAMLVYDPAHRISAKRALQQNYLRDFH

SEQ ID NO: 14 - protein, 298 aa, artificial
  CDKCONF1
MENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFSHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 15 - protein, 297 aa, artificial
  CDKCONF2
MEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFSHSRRVLHRDLKPQNLLIDDKGTIKLADFGLARAFGIPIRVYTHEVVTLWYRSPEVLLGCARYSTPVDIWSIGCIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 16 - protein, 305 aa, artificial
  CDKCONF3
MDMFQKVEKIGEGTYGVVYKAKNRETGQLVALKKIRLDLEMEGVPSTAIREISLLKELKHPNIVRLLDVVHNERKLYLVFEFLSQDLKKYMDSTPGSELPLHLIKSYLFQLLQGVSFSHSHRVIHRDLKPQNLLINELGAIKLADFGLARAFGVPLRTYTHEVVTLWYRAPEILLGCKFYTTAVDIWSIGCIFAEMVTRKALFPGDSEIDQLFRIFRMLGTPSEDTWPGVTQLPDYKGSFPKWTRKGLEEIVPNLEPEGRDLLMQLLQYDPSQRITAKTALAHPYFSSPEPSPAARQYVLQRFRH

SEQ ID NO: 17 - protein, 303 aa, artificial
  CDKCONF4
MATSRYEPVAEIGVGAYGTVYKARDPHSGHFVALKSVRVPNGGGGGGGLPISTVREVALLRRLEAFEHPNVVRLMDVSATSRTDREIKVTLVFEHVDQDLRTYLDKAPPPGLPAETIKDLMRQFLRGLDFLHANSIVHRDLKPENILVTSGGTVKLADFGLARIYSYQMALTPVVVTLWYRAPEVLLQCTYATPVDMWSVGCIFAEMFRRKPLFSGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 18 - protein, 292 aa, artificial
  CDKCONF5
MQKYEKLEKIGEGTYGTVFKAKNRETHEIVALKRVRLDDDDEGVPSSALREISLLKELKHKNIVRLHDVLHSDKKLTLVFEFSDQDLKKYFDSSNGDLDPEIVKSFLFQLLKGLGFSHSRNVLHRDLKPQNLLINRNGELKLADFGLARAFGIPVRSYSAEVVTLWYRPPDVLFGCKLYSTSIDMWSAGCIFAELANAGRPLFPGNDVDDQLKRIFRLLGTPTEEQWPSMTKLPDYKPYPMYPATTSLVNVVPKLNATGRDLLQNLLKSNPVQRISAEEALQHPYFSDFSPP

SEQ ID NO: 19 - protein, 326 aa, artificial
  CDKCONF6
MEKDGLSRADQQYESVAEIGEGAYGKVFKARDLKNGGRFVALKRVRVQTGEEGMPLSTIREVAVLRHLETFEHPNVVRLFDVSTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQCSYATPVDLWSVGCIFAEMFRRKPLFRGSSDVDQLGKILDVIGLPGEEDWPRDVALPRQAFHSKSAQPIEKFVTDIDELGKDLLLKSLTFNPAKRISAYSALSHPYFQDLERSKENLDSHLPPSQNTSELNTA

SEQ ID NO: 20 - protein, 346 aa, artificial
  CDKCONF7
MALDVKSRAKRYEKLDFLGEGQFATVYKARDKNTNQIVAIKKIKLGHRSEAKDGINRTALREIKLLQELSHPNIIGLLDAFGHKSNISLVFDFMETDLEVIIKDNSLVLTPSHIKAYMLMTLQGLEYLHQHWILHRDLKPNNLLLDENGVLKLADFGLAKSFGSPNRAYTHQVVTRWYRAPELLFGCRMYGVGVDMWAVGCILAELLLRVPFLPGDSDLDQLTRIFETLGTPTEEQWPDMSSLPDYVTFKSFPGIPLHHIFSAAGDDLLDLIQGLFLFNPSARITATQALKMKYFSNRPGPTPGSQLPRPNSPVETLKEQSNPALAIKRKRTEALEQGGLPKKLIF

SEQ ID NO: 21 - protein, 464 aa, artificial
  CDKCONF8
MDYDFKVKLSSERERVEDLFEYEGSKVGRGTYGHVYKAKRKDGKDDKDYALKQIEGTGISMSASREIALLRELKHPNVISLQKVFLSHADRKVWLLFDYAEHDLWHIIKFHRASKANKKPVQLPRGMVKSLLYQILDGIHYLHANWVLHRDLKPANILVMGEGPERGRVKIADMGFARLFNSPLKPLADLDPVVVTFWYRAPELLLGCRHYTKAIDIWAIGCIFAELLTSEPIFHSRQEDIKTSNPYHHDQLDRIFNVMGFPADKDWEDIKKMPEHSTLMKDFRRNTYTNSSLIKYMEKHKVKPDSKAFHLLQKLLTMDPIKRITSEQAMQDPYFLEDPLPTSDVFAGSQIPYPKREFLTEEEPDDKGDKKNQQQQQGNNHTNGTGHPGNQDSSHTQGPPLKKVRVVPPTTTSGGLIMTSDYQRSNPHAAYPNPGPSTSQPQSSMGYSATSQQPPQYSHQTHRY

SEQ ID NO: 22 - protein, 372 aa, artificial
  CDKCONF9
MAKQYDSVESPFSDEVSKYEKLAKIGQGTFGEVFKARHRKTGQKVALKKVLMENEKEGFPITALREIKILQLLKHENVVNLIEISRTKASPYNRSKGSIYLVFDFSEHDLAGLLSNVLVKFTLSEIKRVMQMLLNGLYYIHRNKILHRDMKAANVLITRDGVLKLADFGLARAFSLAKNSQPNRYTNRVVTLWYRPPELLLGCRDYGPPIDLWGAGSIMAEMWTRSPIMQGNTEQHQLALISQLSGSITPEVWPNVDNYELYEKLELVKGQKRKVKDRLKAYVRDPYALDLIDKLLVLDPAQRIDSDDALNHDFFWSDPMPSDLKGMLSTHLTSMFEYLAPPRRKGSQITQQSTNQSRNPATTNQTEFERVF

SEQ ID NO: 23 - protein, 4 aa, artificial
  Consensus sequence from substrates of CDK
SPXK

(19) United States
(12) Patent Application Publication
(10) Pub. No.: US 2016/0289650 A1
(43) Pub. Date: Oct. 6, 2016

(54) FLUORESCENT PROTEIN BIOSENSORS TO PROBE THE CONFORMATIONAL ACTIVATION OF CDK/CYCLIN KINASES

(71) Applicants: CENTRE NATIONAL DE LA RECHERCHE SCIENTIFIQUE (CNRS), Paris (FR); UNIVERSITE MONTPELLIER 2 SCIENCES ET TECHNIQUES, Montpellier (FR)

(72) Inventors: May Catherine Morris, Mauguio (FR); Gilles Divita, Mauguio (FR); Morgan Pellerano, Aigues-Vives (FR)

(73) Assignees: Centre National de la Recherche Scientifique (CNRS), Paris (FR); Universite Montpellier 2 Sciences et Techniques, Montpellier (FR)

(21) Appl. No.: 14/442,236

(22) PCT Filed: Nov. 13, 2013
(86) PCT No.: PCT/EP2013/073747

§ 371 Date: May 12, 2015

(30) Foreign Application Priority Data
Nov. 13, 2012 (EP) 12306401.6

(51) Int. Cl. C12N 9/12; G01N 33/58; G01N 33/573

(52) U.S. Cl. CPC C12N 9/12; G01N 33/573; C12Y 207/11022; G01N 33/582; G01N 2500/04; G01N 2333/912

(57) ABSTRACT

The present invention relates to compounds comprising a polypeptide comprising an amino acid sequence derived from the sequence of a cyclin-dependent kinase (CDK) and at least one fluorophore coupled to an amino acid of said polypeptide. The invention also relates to said compounds associated with a technology enabling their intracellular delivery. The present invention also relates to the use of such compounds, or of compositions comprising said compounds, for determining if a product is capable of modulating the conformation of a CDK. More particularly, a compound according to the invention is usable for high-content high-throughput screening.

The present invention relates to compounds comprising a polypeptide comprising an amino acid sequence derived from the sequence of a cyclin-dependent kinase (CDK) and at least one fluorophore coupled to an amino acid of said polypeptide. The invention also relates to said compounds associated with a technology enabling their intracellular delivery. The present invention also relates to the use of such compounds, or of compositions comprising said compounds, for determining if a product is capable of modulating the conformation of a CDK.

Cell cycle progression is driven by a family of serine/threonine protein kinases named Cyclin Dependent Kinases (CDK), whose sequential activities promote phosphorylation of key substrates involved in cell growth and division (Malumbres et al., 2005; Obaya et al., 2002; Satyanarayana et al. 2009; Merrick et al., 2010). Cyclin-CDK complexes are formed through association of a CDK with a Cyclin partner, which plays a major role in promoting activation of the CDK by inducing significant conformational changes, in defining substrate specificity, and in targeting the heterodimeric complex to well-defined subcellular locations (Jeffrey et al., 1995; Morgan et al., 1997; Morris et al. 2002; Lolli et al., 2010). The kinase activity of CDK and/or Cyclin-CDK complexes is primarily conditioned by formation of the Cyclin-CDK complex, and thus expression of either counterpart. This heterodimeric complex is then further regulated by several phosphorylations on the CDK that either inhibit or promote its complete activation (Morgan et al., 1997). Additional regulatory proteins are known to regulate CDK and/or Cyclin-CDK complex activity, such as the INK4 family and the Cip/Kip family of CM (Cyclin-dependent Kinase Inhibitors).

The conformation of the CDK active site changes dramatically upon binding of the Cyclin partner, and this conformational change is directly related to the kinase activity of the CDK. The active site of the CDK lies in a cleft between the two lobes of the kinase. ATP binds deep within a pocket located in close proximity to the catalytic cleft and its phosphate is oriented outwards. Protein substrates bind the active site cleft. In their monomeric, inactive form, CDK does not bind substrates with sufficiently high affinity because the entrance of the catalytic site is blocked by a flexible loop, known as the activation segment and called the T-loop. Upon binding of the Cyclin partner, the T-loop moves out of the catalytic site entrance and no longer blocks the substrate binding site (Jeffrey et al., 1995).

One of the many causes of aberrant CDK activity in cancer cells is the overexpression of the CDK or of the cyclin subunit or the hyperactivation of the CDK associated with—mutations in CDK1, CDK2 or CDK4 (Malumbres et al., 2001; Malumbres et al., 2007).

CDK activities are frequently altered in human cancers, and contribute to sustain abnormal proliferation in cancer cells (Lapenna et al., 2009; Malumbres et al., 2009). More particularly, aberrant CDK activities have been reported in a wide range of cancers including breast, ovarian, prostate, colorectal and lung cancer, lymphoma, myeloma and sarcoma (Harwell et al., 2004; Ekberg et al., 2005; Husdal et al., 2006; Suzuki et al., 2007; Kim et al., 2009).

Despite the oncological relevance, prognostic value and pharmacological attractivity of CDK/Cyclins, approaches currently employed to probe their status remain indirect and invasive, essentially relying on to antigenic and proteomic approaches. Likewise strategies employed in drug discovery for screening compound libraries to identify novel inhibitors of CDK/Cyclins rely on activity-based assays which are somewhat lengthy and tedious, and have essentially yielded compounds that bind the nucleotide pocket of these kinases. However, identification of novel inhibitors of these kinases which do not target the ATP-binding pocket or the catalytic site remains limited, due to a lack of strategies to probe molecules with different mechanisms of action.

Thus, there is still an urgent need for innovative sensing technologies to setup assays for selective identification of inhibitors with defined mechanisms of action that could be used as protein biosensors and interfere with molecules able to modulate the conformational activation of CDK in vitro through the interaction with the T-loop of CDKs. Moreover, drug discovery programs require sensitive means of characterizing the efficacy of candidate inhibitors in a physiological context, and of studying their influence on target behavior in healthy and pathological cells.

The inventors have designed a compound, comprising a polypeptide sequence derived from cyclin-dependent kinases, and a fluorophore. The fluorescence of the compound of the invention increases when its conformation changes, in a reversible fashion. The compound can be used to screen for modulators of the conformation of CDK, and thus in particular for inhibitors of the activity of CDK, through fluorescence imaging. A compound according to the invention is usable as a sensor of conformational modulation of a CDK, however the modulation of CDK conformation may not be restricted to a conformational activation. Moreover the compound does not need to have catalytic activity; in fact no catalytic activity is required for the compound to serve as a sensor of conformational modulation; conformational dynamics of the activation segment are required. Indeed, the modulation of CDK conformation may be due to the binding of a Cyclin partner, or of a substrate, or a cofactor, or yet another protein partner.

The inventors have set up methods that allows for the detection of subtle differences in CDK conformation, and thus CDK activity, for example upon contact with a product or drug candidate, in a standardized and sensitive, yet non-destructive fashion. Those compounds and methods afford direct readout of the variation of CDK conformation and/or activity for drug discovery strategies.

The compound of the invention is based on the strong fluorescence enhancement exhibited by fluorophores, particularly environmentally-sensitive dyes, when their exposure to their immediate environment is modified. The compound of the invention undergoes conformational changes that modify substantially the immediate environment of the fluorophore upon contact with a product or drug capable of modifying the T-Loop conformation of the CDK from which the compound is derived. The emitted fluorescence of the compound thus varies with its conformation, and particularly increases substantially when the T-Loop is displaced from the catalytic site, for example when the cyclin binds the CDK, that is when the CDK is in an active conformation. These variations are reversible, and thus the compound of the invention can be used in methods such as real-time analysis of CDK activity or competition kinetics.

This new technology is therefore a useful tool to monitor activation or inhibition of cyclin-dependent kinases in vitro, and provides information which cannot be obtained through antigenic or radioactive approaches, in a sensitive, specific and non-invasive fashion. Moreover, this technology is directly applicable to screens of small compound libraries to identify novel inhibitors of cyclin-dependent kinases that affect the conformational step of their activation. Furthermore, coupling of CDKCONF to a technology for intracellular internalisation such as the cell-penetrating peptide technology allows for the direct application of this biosensor in living cultured mammalian cells, and further in vivo. An adequate formulation of a compound according to the invention, with cell-penetrating peptides, allows its optimal delivery into mammalian cells, which further allows in particular for its application to high content cell-based screening assays.

### DETAILED DESCRIPTION OF THE INVENTION

Each amino acid is herein represented according to the IUPAC amino-acid abbreviation, such as follows:

TABLE 1

| Amino-acid or amino- | | |
|---|---|---|
| acid residue | Abbreviation | Abbreviation |
| Alanine | Ala | A |
| Arginine | Arg | R |
| Asparagine | Asn | N |
| Aspartic acid | Asp | D |
| (Aspartate) | | |
| Cysteine | Cys | C |
| Glutamine | Gln | Q |
| Glutamic acid | Glu | E |
| (Glutamate) | | |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Aspartic acid or | Asx | B |
| Asparagine | | |
| Glutamine or | Glx | Z |
| Glutamic acid | | |
| Any amino acid | Xaa | X |

The present invention first relates to compounds comprising a polypeptide and at least one fluorophore, wherein:
a) the amino acid sequence of said polypeptide comprises the sequence V/IVTXWYRXPXI/V/LL (SEQ ID No. 1) and is derived from the amino acid sequence of at least one sequence of a protein from the CDK family, and
b) said at least one fluorophore is coupled to an amino-acid residue of said polypeptide, wherein the position of said amino acid within the sequence of said polypeptide is chosen from the group consisting of: position 6 to position 36, wherein position 1 within the sequence of said polypeptide is defined as the position of the first amino acid of said sequence V/IVTXWYRXPXI/V/LL (SEQ ID No. 1).

Based on sequence similarity, the human genome contains 21 genes encoding CDKs and five additional genes encoding a more distant group of proteins known as CDK-like (CDKL) kinases (Malumbres et al., 2009). The current nomenclature for CDKs includes 11 classical CDKs (CDK1-11), two newly proposed family members (CDK12 and 13) and additional proteins whose names are based on the presence of a cyclin binding element or sequence relationship. It is known that CDK activity depends on association with a cyclin-like regulatory subunit (Malumbres et al., 2009; Morris et al. 2002; Jeffrey et al. 1995). By proteins chosen from the “group of the CDK family” it is herein referred to a serine/threonine kinase protein with sequence and structural similarity to CDK2 (SEQ ID No. 3 and PDB No 1B38), such as described in Lolli G. et al (2010).

From a structural point of view CDKs present a characteristic bilobe kinase fold, with an N-terminal lobe that bears an ATP-binding pocket and a C-terminal-lobe, between which is positioned the catalytic cleft where the substrate binds, and whose access is regulated by a flexible activation segment, termed the T-loop. Secondary structure elements are very well conserved between CDKs with minimal differences in terms of their length, and structural deviations cluster in a few regions that constitute interacting surfaces differentially involved in recognition of specific binding partners (Lolli et al., 2010). Within the N-terminal lobe, loops Beta 3-alpha C and beta 4-beta 5 together with alpha helix C constitute the common CDK/Cyclin interface.

Activation of cyclin-dependent kinases is a complex process, initiated by the association of the CDK with a cyclin, followed by a series of regulatory phosphorylation/dephosphorylation steps. CDK-Cyclin complex assembly is a two-step mechanism, which has been well characterized for CDK2-Cyclin A (Morris et al., 2002), involving a first rapid protein/protein interaction, which is identical for all CDK-cyclin partners, and a second step, which corresponds to a slow isomerization of the CDK, which is essential for its full activation. This cyclin-induced conformational reorganization of the CDK has been well characterized from a structural point of view for CDK2-cyclinA. It involves a reorientation of the ATP-binding pocket of CDK2, thereby aligning it with the catalytic cleft in a position which is appropriate for transfer of phosphate onto the substrate, as well as a positional switch of an activating segment termed the T-loop by 20 A, which opens the catalytic cleft, thereby providing access for the substrate, and exposes Thr160 for subsequent phosphorylation by the CDK-Activating Kinase CAK. This phosphorylation further displaces the T-loop and stabilizes it in a position which contributes to full activation of the CDK/Cyclin complex by generating a fully accessible substrate-binding site, and favouring substrate binding which overall ameliorates catalysis (De Bondt H L et al., 1993; Jeffrey P D et al. 1995, Russo A A et al., 1996, Brown N R et al., 1999).

Proteins of the CDK family according to the invention can be from any known species, such as but not limited to: animals, preferably vertebrates, plants or microorganisms.

“Amino acid sequence” and “sequence” will be employed indifferently in the present specification. “Amino acid” and “amino acid residue” will also be employed indifferently. By “sequence derived from the amino acid sequence of at least one peptide” it is herein referred to an amino acid sequence having at least 70% identity with the reference amino acid sequence, preferably at least 80% identity, more preferably at least 90% identity, more preferably at least 95% identity, and most preferably at least 98% identity with the amino acid sequence of said peptide.

As used herein the term “identity” herein means that two amino acid sequences are identical (i.e. on an amino acid basis) over the window of comparison. The term “percentage of sequence identity” is calculated by comparing two optimally aligned sequences over the window of comparison, in which the amino acid sequence to be compared can contain additions or deletions with respect to the reference sequence for optimal alignment between those two sequences. The percentage identity is calculated by determining the number of positions at which the identical amino acid residues occur in both sequences to yield the number of matched positions, dividing the number of matched positions by the total number of positions in the window of comparison (i.e. the window size) and multiplying the result by 100 to yield the percentage of sequence identity. The percentage of sequence identity of an amino acid sequence can also be calculated using BLAST software with the default or user defined parameter.

The amino acid sequences are read from the N-Terminal extremity to the C-Terminal extremity of said amino acid sequence of the polypeptide. Therefore the amino acid in position 1, i.e. the first amino acid, of the sequence V/IVTXWYRXPXI/V/LL (SEQ ID No. 1) is the amino acid at the N-terminal extremity of the sequence and is either a Valine or an Isoleucine residue.

The T loop of a polypeptide according to the invention, which is defined as the activation segment which blocks or opens the catalytic site of the CDK, should preferably be mobile, dynamic, and its conformational change upon interaction with a modulator should lead to significant changes in the fluorescence of the coupled fluorophore.

According to the invention, the sequence of the polypeptide according to the invention has at least 70% identity, preferably at least 80% identity, preferably at least 90% identity, more preferably at least 95% identity and still more preferably at least 98% identity with the peptide sequence of at least one protein from the CDK family.

In an embodiment, the sequence of the polypeptide according to the invention has at least 70% identity, preferably at least 80% identity, preferably at least 90%, more preferably 95% identity, and even more preferably at least 98% identity with the peptide sequence of at least one protein from the list consisting of:
a) the human proteins CDK1 (SEQ ID No. 2), CDK2 (SEQ ID No. 3), CDK3 (SEQ ID No. 4), CDK4 (SEQ ID No. 5), CDK5 (SEQ ID No. 6), CDK6 (SEQ ID No. 7), CDK7 (SEQ ID No. 8), CDK8 (SEQ ID No. 9), CDK9 (SEQ ID No. 10) and CDK10 (SEQ ID No. 11),
b) the Saccharomyces cerevisiae protein CDC28 (SEQ ID No. 12),
c) the Schizosaccharomyces pombe protein CDC2 (SEQ ID No. 13).

In a preferred embodiment, the sequence of the polypeptide according to the invention has at least 70% identity, preferably at least 80% identity, preferably at least 90%, more preferably at least 95% identity and still more preferably at least 98% identity with the peptide sequence of CDK2 (SEQ ID No. 3).

In another embodiment, the sequence of the polypeptide according to the invention has at least 70% identity, preferably at least 80% identity, preferably at least 90%, more preferably at least 95% identity and still more preferably at least 98% identity with the peptide sequence of a human protein chosen among the following: CDK1 (SEQ ID No. 2), CDK3 (SEQ ID No. 4), CDK4 (SEQ ID No. 5), CDK5 (SEQ ID No. 6), CDK6 (SEQ ID No. 7), CDK7 (SEQ ID No. 8), CDK8 (SEQ ID No. 9), CDK9 (SEQ ID No. 10) and CDK10 (SEQ ID No. 11), wherein the modification of said amino acid sequence is chosen by analogy with the amino acid sequence of CDK2 (SEQ ID No 3) and the modification of amino acid sequence of CDK2 (SEQ ID No 14), as it is known that the proteins belonging to the “group of the CDK family” exhibit strong sequence and 3D structure similarities to CDK2.

In a preferred embodiment, the sequence of the polypeptide according to the invention has at least 70% identity, preferably at least 80% identity, preferably at least 90%, more preferably at least 95% identity and still more preferably at least 98% identity with the peptide sequence of at least one protein from the list consisting of: the human proteins CDK1 (SEQ ID No. 2), CDK2 (SEQ ID No. 3), CDK3 (SEQ ID No. 4), CDK4 (SEQ ID No. 5), CDK5 (SEQ ID No. 6), CDK6 (SEQ ID No. 7), CDK7 (SEQ ID No. 8), CDK8 (SEQ ID No. 9), CDK9 (SEQ ID No. 10) and CDK10 (SEQ ID No. 11), the Saccharomyces cerevisiae protein CDC28 (SEQ ID No. 12), the Schizosaccharomyces pombe protein CDC2 (SEQ ID No. 13), wherein said polypeptide comprises at most two amino acid residue comprising a thiol group; within the sequence of said polypeptide, the position of each of at most two amino acid residue comprising a thiol group is chosen from the group consisting of position 6 to position 36, wherein position 1 of the amino acid sequence of said polypeptide is the position of the first amino acid of said sequence V/IVTXWYRXPXI/V/LL (SEQ ID No. 1).

The polypeptide according to the invention is obtainable with an appropriate expression system or any techniques known to/by the person skilled in the art, such as chemical synthesis. Production of the polypeptide according to the invention may be done for example in expression systems derived from bacteria, yeast, baculovirus, insect, and mammalian cells, or in cell-free expression systems, reviewed in Higgins et al, in Baneyx F et al, and in Atherton et al.

In a particular embodiment, the invention relates to compounds comprising a polypeptide and at least one fluorophore, the sequence of said polypeptide comprises at most two amino acid residues comprising a thiol group, wherein the position within the sequence of said polypeptide of said at most two amino acid residues comprising a thiol group is chosen from the group consisting of: position 6 to position 36.

In a more particular embodiment, the invention relates to compounds comprising a polypeptide and at least one fluorophore, the sequence of said polypeptide comprises at most two amino acid residues comprising a thiol group, said amino acid residues being preferably cysteine residues.

In an even more particular embodiment, the invention relates to compounds comprising a polypeptide and at least one fluorophore, the sequence of said polypeptide comprising two cysteine residues.

In an even more particular embodiment, the invention relates to compounds comprising a polypeptide and one fluorophore, the sequence of said polypeptide comprising two cysteine residues.

In another particular embodiment, the invention relates to compounds comprising a polypeptide comprising an amino acid sequence derived from a sequence chosen in the list consisting of: SEQ ID No. 14, SEQ ID No. 15, SEQ ID No. 16, SEQ ID No. 17, SEQ ID No. 18, SEQ ID No. 19, SEQ ID No. 20, SEQ ID No. 21 and SEQ ID No. 22.

In a more particular embodiment, the invention relates to compounds comprising a polypeptide the amino acid sequence of which is derived from a sequence chosen in the list consisting of: SEQ ID No. 14, SEQ ID No. 15, SEQ ID No. 16, SEQ ID No. 17, SEQ ID No. 18, SEQ ID No. 19, SEQ ID No. 20, SEQ ID No. 21 and SEQ ID No. 22, with said sequence comprising at most two amino acid residues comprising a thiol group, said amino acid residues being preferably cysteine residues.

In an even more particular embodiment, the invention relates to compounds comprising a polypeptide—the amino acid sequence of which is chosen in the list consisting of: SEQ ID No. 14, SEQ ID No. 15, SEQ ID No. 16, SEQ ID No. 17, SEQ ID No. 18, SEQ ID No. 19, SEQ ID No. 20, SEQ ID No. 21 and SEQ ID No. 22.

In an even more particular embodiment, the invention relates to a compound comprising a polypeptide comprising or having the amino acid sequence SEQ ID No. 14.

In an even more particular embodiment, the invention relates to a compound comprising a polypeptide comprising or having the amino acid sequence SEQ ID No. 17.

In another particular embodiment, the present invention relates to a compound comprising a polypeptide and two fluorophores, with each of the fluorophores being coupled to an amino acid residue of said peptide.

According to the invention, the fluorophore coupled to an amino-acid residue of the polypeptide is any fluorescent molecule. By “fluorophore” it is herein meant a molecule capable of re-emitting light upon light excitation. In most cases, emitted light has a longer wavelength, and therefore lower energy, than the absorbed light. The emitted light may also be of the same wavelength as the absorbed light, termed “resonance fluorescence”. Fluorophores typically contain several combined aromatic groups, or plane or cyclic molecules with several it bonds.

According to the invention, the fluorophore is for example chosen from Xanthene, Cyanine, Naphthalene, Coumarin, Oxadiazole, Pyrene, Oxazine, Acridine, Arylmethine or Tetrapyrrole derivatives. The fluorophore chosen from the Xanthene derivatives is for example fluorescein, rhodamine, Oregon green, eosin, or Texas red. The fluorophore chosen from the Cyanine derivatives is for example: cyanine, indocarbocyanine, oxacarbocyanine, thiacarbocyanine, or merocyanine. The fluorophore chosen from the Naphthalene derivatives is for example dansyl or prodan. The fluorophore chosen from the Oxadiazole derivatives is for example pyridyloxazole, nitrobenzoxadiazole or benzoxadiazole. The fluorophore chosen from the Pyrene derivatives is for example cascade blue. The fluorophore chosen from the Oxazine derivatives is for example: Nile red, Nile blue, cresyl violet, or oxazine 170. The fluorophore chosen from the Acridine derivatives is for example proflavin, acridine orange, or acridine yellow. The fluorophore chosen from the Arylmethine derivatives is for example auramine, crystal violet, or malachite green. The fluorophore chosen from the Tetrapyrrole derivatives is for example porphin, phtalocyanine, or bilirubin.

Preferably, the fluorophore according to the invention is chosen in the list consisting of Hydroxycoumarin, Aminocoumarin, Methoxycoumarin, Cascade Blue, Pacific Blue, Pacific Orange, Lucifer yellow, NBD, R-Phycoerythrin (PE), PE-Cy5 conjugates, PE-Cy7 conjugates, Red 613, PerCP, TruRed, FluorX, Fluorescein, BODIPY-FL, Cy2, Cy3, Cy3B, Cy3.5, Cy5, Cy5.5, Cy7, TRITC, X-Rhodamine, Lissamine Rhodamine B, Texas Red, Allophycocyanin (APC), APC-Cy7 conjugates.

According to another particular embodiment, the invention relates to a compound comprising a polypeptide and at least one fluorophore, wherein said at least one fluorophore is coupled to a cysteine residue.

The inventors have found that the change in fluorescence emission upon interaction of the polypeptide with a modulator that affects or induces a change of conformation of the polypeptide according to the invention is more easily monitored and allows for a more accurate and sensitive measure of said binding when using fluorophores that are environment-sensitive dyes.

In an embodiment, at least one fluorophore is an environment-sensitive dye. The terms “environment-sensitive dye”, “environment-sensitive probe”, “solvatochromic dye”, “solvatochromic probe” are herein interchangeable. By environment-sensitive dye, it is herein meant a fluorophore the properties of which change, for example intensity, half-life, and excitation or emission spectra, in a measurable manner upon a change in the fluorophore environment. Preferably, by environment-sensitive dye, it is herein meant a fluorophore, the intensity or emission spectrum of which changes upon a change in its environment. According to the invention, the change in the fluorophore environment may be due to at least one of a variety of different environmental factors which affect the local polarity, such as a more hydrophobic or hydrophilic environment. Environment-sensitive probes have been reviewed in Loving et al. (2010).

Environment-sensitive dyes are well known by the skilled person and may include for example any dye that contains an electron-donating and an electron-accepting group at opposite ends of the aromatic system. According to the invention, the environment-sensitive dye is for example, without restriction to those examples, Cascade Yellow, prodan, Dansyl, Dapoxyl sulfonic acid, NBD, PyMPO, Pyrene, diethylaminocoumarin, SYPRO Orange dye, SYPRO Red dye, nile red, CPM (7-Diethylamino-3-(4′-Maleimidylphenyl)-4-Methylcoumarin), DCDHF (2,7-Dichlorodihydrofluorescein diacetate), fluorophore from the BODIPY family of dyes (boron-dipyrromethene family of dyes).

In a particular embodiment, the present invention relates to a compound comprising a polypeptide according to the invention and a fluorophore, wherein said fluorophore is chosen in the group consisting of solvatochromic probes, wherein said solvatochromic probe is chosen among the following: Cy3, PRODAN, NBD, Coumarin derivatives, Oxazine derivatives, dansyl derivatives, dimethylamino-phthalimide/naphtalimides, merocyanines, Dapoxyl® derivatives, PyMPO.

According to the invention, the fluorophore is coupled to specific functional groups of the amino acid residues of the polypeptide of the invention, for example but not limited to amino, carboxyl, thiol or azide groups. In a preferred embodiment, the fluorophore is coupled to a thiol group of an amino-acid residue. In a more preferred embodiment, the fluorophore is coupled to a thiol group of a cysteine residue. Coupling the fluorophore to an amino acid functional group is a technique well known by the skilled person, and may involve chemical reactions such as for example amine coupling of lysine amino acid residues (typically through amine-reactive succinimidyl esters), sulfhydryl coupling of cysteine residues (via a sulfhydryl-reactive maleimide) or initiated free radical reactions.

The inventors have found that when the polypeptide sequence according to the invention comprises only two cysteine residues within a region comprised between the amino-acid residue in position 6 to 36, wherein position 1 corresponds to the first amino-acid residue of the sequence SEQ ID No. 1, the coupling of the fluorophore to the polypeptide is facilitated. More precisely, when the sequence of polypeptide according to the invention contains at the most two amino-acid residues with a thiol group, preferably cysteine, and when each of those two amino-acids residues with a thiol group has a position chosen from the list consisting of positions 6 to 36, wherein position 1 correspond to the first amino-acid residue of said sequence V/IVTXWYRXPXI/V/LL (SEQ ID No. 1), the coupling of the polypeptide with the fluorophore of the invention, preferably Cy3, is facilitated.

More preferably, the polypeptide sequence of the compound according to the invention has the sequence chosen in the list consisting of: SEQ ID No. 14, SEQ ID No. 15, SEQ ID No. 16, SEQ ID No. 17, SEQ ID No. 18, SEQ ID No. 19, SEQ ID No. 20, SEQ ID No. 21 or SEQ ID No. 22 and is characterized in that at least one fluorophore is Cy3 and is coupled to the polypeptide on a cysteine residue. Even more preferably, the polypeptide sequence of the compound according to the invention has the sequence chosen in the list consisting of: SEQ ID No. 14, SEQ ID No. 15, SEQ ID No. 16, SEQ ID No. 17, SEQ ID No. 18, SEQ ID No. 19, SEQ ID No. 20, SEQ ID No. 21 or SEQ ID No. 22 and is characterized in that two fluorophores are coupled to the polypeptide on cysteine residues and are Cy3 fluorophores.

More preferably, the polypeptide sequence of a compound according to the invention has the sequence SEQ ID No. 14 and is characterized in that at least one fluorophore is Cy3 and is coupled to the polypeptide on a cysteine residue.

Even more preferably, the polypeptide sequence of a compound according to the invention has the sequence SEQ ID No. 14 and is characterized in that at the fluorophore Cy3 is coupled to two cysteine residues of said polypeptide.

The compound of the invention may be prepared to allow its direct use in a cell, in cell culture, including tissue culture, or in animal and/or human tissues, originating for example from biopsies. Thus, in an embodiment, the compound of the invention further comprises means to penetrate the cell membrane. In the list consisting of the means to penetrate cell membrane known by the skilled person, one can mention cell penetrating peptides. As used herein, the terms “cell penetrating peptide”, “cell-permeable peptides”, “protein-transduction domains (PTD)”, “membrane-translocation sequences (MTS)” are equivalent. A cell-penetrating peptide is defined as a short (5 to 40 aa) polycationic or amphiphilic peptide which can readily cross biological membranes and capable of facilitating cellular uptake of various molecular cargos, in vitro and/or in vivo. As used herein, the term “molecular cargo” refers to a molecule in the list consisting of chemical molecules, peptides, polypeptides, proteins or nucleotides.

In a preferred embodiment, the compound of the invention further comprises a cell penetrating peptide (CPP) sequence. Preferably, the cell penetrating peptide according to the invention is capable of facilitating cellular uptake of peptides, polypeptides or proteins. More preferably, the cell penetrating peptide according to the invention is capable of facilitating cellular uptake of peptides of a more than 5 Amino-acids and up to at least 500 kDa (Kurzawa et al. 2010; Morris et al., 2001). Such cell penetrating peptides are well known by the skilled person, and are described thoroughly in Grdisa et al., 2011) or Matjaz et al., 2005, Morris et al., 2008, Fonseca et al., 2009, Heitz et al., 2009). Cell penetrating peptides (CPP) are known by persons skilled in the art (Morris et al. 2008). Examples of CPP are the following: the third alpha helix of Antennapedia homeobox and the transactivating regulatory domain of TAT from HIV, PEP, Pep1 and CADY (Morris et al, 2001, Kurzawa et al, 2010), Transportan, VP22, amphipathic peptides such as Pep1 and CADY2, polycationic peptides and oligoarginines

In a particular embodiment, the invention relates to a compound comprising a polypeptide, at least one fluorophore and a cell penetrating peptide, said cell penetrating peptide and polypeptide being preferably associated through non-covalent interactions. In another particular embodiment, the invention relates to a compound comprising a polypeptide, at least one fluorophore and a cell penetrating peptide, said cell penetrating peptide and polypeptide being associated through covalent interactions. In a more particular embodiment, the cell penetrating peptide is located on the N-terminal extremity of the polypeptide. In another particular embodiment, the invention relates to a compound comprising a polypeptide, one fluorophore and a cell penetrating peptide, said cell penetrating peptide and polypeptide being associated through covalent interactions.

According to the invention, the compound may further comprise a protein tag. Protein tags are well known by the skilled person and may for example be chosen in the list consisting of Isopeptag, BCCP, Myc-tag, Calmodulin-tag, FLAG-tag, HA-tag, His-tag, Maltose binding protein-tag, Nus-tag, Glutathione-S-transferase-tag, Green fluorescent protein-tag, Thioredoxin-tag, S-tag, Softag 1, Softag 3, Strep-tag, SBP-tag, Ty tag, V5 tag or TC tag.

The present invention also relates to a compound comprising a polypeptide and at least one fluorophore used as a conformation-sensitive biosensor.

The fluorescence emitted by the compound of the invention varies with the conformational state of a CDK, for example the fluorescence emitted is enhanced when the T-loop domain is in a conformation which allows access to the catalytic cleft, that is, when the substrate-binding domain and of said CDK is accessible. Thus, a compound according to the invention may be useful for example for screening, particularly high throughput screening, to identify modulators of the conformation of at least one CDK.

The inventors have discovered that the compound of the invention can be used to monitor the changes of conformation of at least one CDK, particularly the changes in the conformation of the T-Loop of at least one CDK. The compound according to the invention emits a fluorescence that changes, for example in intensity or wavelength, depending on the significance of the conformational change induced in the compound and inherently on the activation state of said CDK.

In another aspect of the invention, the compound according to the invention is used to monitor changes in a CDK conformation, for real-time analysis of CDK activity or in competition kinetics analysis. In a preferred embodiment, the compound of the invention is used a conformation-sensitive biosensor.

In another embodiment, the invention also relates to methods for determining if a product is capable of modulating the conformation of at least one CDK, said method comprising the steps of:
a) providing at least one compound according to the invention,
b) contacting said compound with said product,
c) illuminating said compound and product with an excitation light,
d) determining the fluorescent signal emitted by said compound,
e) comparing said fluorescent signal with a reference fluorescent signal, and
f) determining from the comparison of step e) if said product is capable of modulating the conformation of at least one CDK.

According to the invention, contacting the compound of the invention with said product is performed, for example by contacting the compound of the invention with solutions, extracts, particularly cell extracts, or any type of sample containing said product.

The skilled man will easily adapt the intensity and wavelength of the excitation light of step c), for example depending on the fluorophore of the compound according to the invention of step a). In an embodiment, the illumination of step c) is performed at a wavelength corresponding to the excitation wavelength of the fluorophore of the compound according to the invention of step a). Various light sources may be used to provide for the excitation light, including lasers, photodiodes, and lamps, preferably xenon arc lamps and mercury-vapor lamps.

According to the invention, determining the fluorescence in step d) can be achieved by any technique and using any appropriate apparatus known in the art. Any fluorimeter or device adapted to measure the properties of emitted light, preferably fluorescence light may be used to determine the fluorescence of step d).

As used herein, “determining the fluorescent signal” or “determining the emitted fluorescence” or “determining the fluorescence level” refers to measuring the properties of the emitted fluorescence, such as for example measuring the wavelength spectrum, intensity or half-life of the emitted fluorescence. In an embodiment, determining the fluorescence emitted is achieved by measuring the wavelength spectrum of the emitted fluorescence. In another embodiment, determining the fluorescence emitted is achieved by measuring the intensity of the emitted fluorescence.

According to the invention, comparing the emitted fluorescence in step e) means comparing the properties of the fluorescent signal of step d) and the properties of the fluorescence reference. In an embodiment, comparing the fluorescence in step e) means comparing the wavelength spectrum of the emitted fluorescence of step d) and the wavelength spectrum of the fluorescence reference. In another embodiment, comparing the fluorescence in step e) means comparing the intensity of the emitted fluorescence of step d) to the intensity of the fluorescence reference at a chosen wavelength.

According to the invention, the reference fluorescence is a predetermined measure of fluorescence, obtained with a product known to be a modulator of at least one CDK conformation. In an embodiment, the reference fluorescence is a predetermined measure of fluorescence obtained with a product known to be a modulator of the conformation of at least one CDK.

The invention thus also relates to the use of at least one compound according to the invention for selecting modulators of conformation of at least one cyclin-dependent-kinase.

The invention furthermore relates to a method for screening a plurality of products for their ability to modulate the conformation of at least one CDK, said method comprising the steps of:
a) providing at least one compound according to the invention,
b) contacting said compound with at least one of said products,
c) illuminating said compound and said at least one product with an excitation light,
d) determining the fluorescent signal emitted by said compound,
e) comparing said fluorescent signal with a reference fluorescent signal, and
f) determining, from the comparison of step d), the ability of said at least one product to modulate the conformation of at least one CDK.

Cyclin-dependent kinases are mostly abnormally activated in a wide range of diseases, particularly in a wide range of cancers. Different types of molecules may be able to inhibit CDK activity, and some of them act by binding to the CDK and modifying the CDK conformation, thus altering its activity. The compound of the invention may thus be useful for example for screening, particularly high throughput screening, for inhibitors of the activity of at least one CDK.

The invention thus also relates to the use of a compound according to the invention for the screening of a plurality of products for their ability to modulate the conformation of at least one CDK, and for selecting modulators of conformation of at least one cyclin-dependent-kinase.

According to the invention, the “reference fluorescence” or the “reference fluorescent signal” is a predetermined measure of fluorescence, obtained with a product known to be an inhibitor of at least one CDK activity, preferably with a product known to inhibit at least one CDK activity by modifying its conformation.

In an embodiment, the reference fluorescence is a predetermined measure of fluorescence obtained with a product known to be an inhibitor of one CDK activity, preferably with a product known to inhibit CDK activity by modifying its conformation.

In a particular embodiment, the invention relates to a method for screening a plurality of products for their ability to modulate the conformation of at least one CDK, wherein said reference fluorescent signal is the signal obtained when using the C4 peptide (Gondeau et al. 2005) as a reference peptide.

In another particular embodiment, the invention relates to a method for screening wherein at least the steps b), c) and d) are performed in miniaturized adapted conditions. Preferably, at least steps b), c) and d) are performed on plates, for example on 384-well plates.

In another particular embodiment, the method for screening comprises an additional step of eliminating compounds which are auto-fluorescent at a wavelength close to the wavelength used for detecting variations in fluorescence of the probe coupled to the CDK, associated with the conformational change of the CDK and/or for the detection of reference signal.

In another particular embodiment, the method for screening comprises an additional step of performing steps b), c) and d) on selected compounds, for a second round of screening. As an example, a first round of screening may be performed in miniaturized adapted conditions, and a second round of screening may be performed “manually” on the compounds selected during the first round of screening.

In another particular embodiment, the method for screening comprises an additional step of determining the IC50 of the complexes formed by compounds and the CDKCONF biosensor.

In another particular embodiment, the method for screening comprises an additional step of determining the ability of the compounds to inhibit the kinase activity of a CDK, for example by performing an in vitro kinase assay.

In another particular embodiment, the method for screening comprises an additional step of determining the ability of the compounds to inhibit the viability of target cells, for example by performing an in vivo proliferation assay on identified cell lines. As an example, a target cell line might be a tumoral cell line.

In another particular embodiment, the method for screening comprises an additional step of determining the dissociation constant (Kd) of the selected compounds for at least one CDK or for at least one CDK/Cyclin complex.

In another particular embodiment, the method for screening comprises an additional step of determining the auto-fluorescent properties of the selected compounds.

In another particular embodiment, the method for screening comprises an additional step of determining the binding site of the selected compounds to the CDK.

The invention also relates to a method for the in vitro diagnosis in a subject of a disease associated with alteration of at least one cyclin, said method comprising the steps of:
a) providing at least one compound according to the invention,
b) contacting said compound with a biological sample from said subject,
c) illuminating said biological sample and compound with an excitation light,
d) determining fluorescence emitted by said compound,
e) comparing said fluorescence with a fluorescence reference, and
f) determining from the comparison of step e) if there is alteration of at least one cyclin.

As used herein, the term “a disease associated with alteration of at least one cyclin” means that said disease is associated with the alteration of the structure and/or the quantity of at least one cyclin. In particular, said alteration can be cyclin overexpression, when compared to corresponding normal expression level of said cyclin.

According to the invention, the above or under normal expression level is determined by comparison of the test value with a reference value. The reference value according to the invention is for example a value obtained by the present method with a biological sample wherein the cyclin or cyclin-CDK complex level and/or activity is normal, such as for example non transformed cell lines, for example normal diploid fibroblast, preferably the HS68 cell line (ATCC code HTB-138). The terms “individual”, “subject”, and “host” are used herein interchangeably and refer to any subject from whom diagnosis is required, particularly humans. Other subjects may include cattle, dogs, cats, guinea pigs, rabbits, rats, mice horses, and the like. In some preferred embodiments, the subject is a human.

As used herein, the term “biological sample” refers to biological material from a subject. The sample assayed by the present invention is not limited to any particular type. Samples include, as non-limiting examples, single cells, multiple cells, tissues, tumors, biological fluids, biological molecules, or supernatants and/or extracts of any of the foregoing. Examples of tissues include tissue removed for biopsy, tissue removed during resection, blood, serum, plasma, sputum, lymph tissue, lymph fluid, cerebrospinal fluid, mucous, skin, saliva, gastric secretions, semen, seminal fluid, tears, spinal tissue or fluid, cerebral fluid, trigeminal ganglion sample, a sacral ganglion sample, adipose tissue, lymphoid tissue, placental tissue, upper reproductive tract tissue, gastrointestinal tract tissue, male genital tissue, fetal central nervous system tissue and stool samples.

The sample used may vary based on the assay format, the detection method and the nature of the tissues, cells or extracts to be assayed. Methods for preparing samples are well known in the art and can be readily adapted in order to obtain a sample that is compatible with the method utilized.

According to the invention, the “reference fluorescence” or the “reference fluorescent signal” is a predetermined measure of fluorescence, obtained from a biological sample where the cyclin and/or the cyclin/CDK complex of interest is known to be normally active.

In an embodiment, the reference fluorescence is a predetermined measure of fluorescence obtained from a reference biological sample from the subject according to the invention, wherein the reference biological sample is known to have cyclin and/or cyclin-CDK complex of interest normally active. Preferably, the reference fluorescence is a predetermined measure of fluorescence obtained from a biological sample from a subject known to have cyclin and/or cyclin CDK complex of interest normally active.

The disregulation of cyclin-dependent kinase level or activation is suspected to contribute to the observed sustained aberrant proliferation of cancer cells, and as such is considered as a hallmark of several diseases. Indeed, the levels of either cyclin or cyclin-dependent kinases, as well as the kinase activity of cyclin-dependent kinases are frequently altered in human cancers. A cyclin-dependent kinase hyperactivation has been reported in a wide range of cancers including breast, ovarian, prostate, colorectal, and lung cancers, as well as lymphoma, myeloma, sarcoma and glioblastoma.

The invention also relates to a method for the in vitro diagnosis of cancer in a subject, comprising the steps of:
a) providing at least one compound according to the invention,
b) contacting said compound with a biological sample from said subject,
c) illuminating said compound and products with an excitation light,
d) determining the fluorescent signal emitted by said compound,
e) comparing said fluorescent signal with a reference fluorescent signal, and
f) diagnosing a cancer in said subject from the comparison of step e) if the fluorescence of step d) is above the reference fluorescent signal.

As used herein, “cancer” refers to primary or metastatic cancers, leukemia or lymphomas, colon cancer, liver cancer, testicular cancer, thymus cancer, breast cancer, skin cancer, oesophagal cancer, pancreatic cancer, prostatic cancer, uterine cancer, cervical cancer, lung cancer, bladder cancer, ovarian cancer, multiple myeloma, melanoma and globlastoma. Preferably, the cancer according to the invention is a cyclin and/or cyclin-CDK complexe associated cancer. By “cyclin and/or cyclin-CDK complex associated cancer” it is herein referred to cancers associated with an hyperactivity of at least one cyclin and/or cyclin-CDK complex.

In an embodiment, the reference fluorescent signal is a predetermined measure of fluorescence, obtained from one or several biological sample from subjects known to have cancer. In a particular embodiment, the reference fluorescent signal is a statistically relevant data obtained from predetermined measures of fluorescence, obtained from several biological samples from subjects known to have cancer.

The invention also discloses a method for evaluating in vitro the therapeutic efficiency of a treatment for a subject, comprising the steps of:
a) providing at least one compound according to the invention,
b) contacting said compound with a biological sample from said subject,
c) illuminating said compound and products with an excitation light,
d) determining the fluorescence signal emitted by said compound,
e) comparing said fluorescence signal with a reference fluorescence signal determined in said subject before the treatment, and
f) determining that said treatment is therapeutically efficient for said subject from the comparison of step e) if the fluorescence of step d) is lower than the fluorescence signal determined in said subject before the treatment.

The invention also relates to kits comprising at least one compound according to the invention and an acceptable carrier and/or an acceptable solvent. In an embodiment, the kit according to the invention further comprises a product known to be a modulator of the conformation of at least one CDK. In another embodiment, the kit according to the invention further comprises a product known to be an inhibitor of the activity of at least one CDK.

As used herein, the term “carrier” refers to a diluent, adjuvant, excipient, or vehicle. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers.

The following examples are provided herein for purposes of illustration only and are not intended to be limiting unless otherwise specified.

### BRIEF DESCRIPTION OF THE DRAWINGS

FIGS. 1A to 1D: Design and engineering the CDKCONF Sensor

FIG. 1A: Structural representation CDK2/cyclinA highlighting the position of the three cysteines. Positions of C118, C177 and C191 in CDK2 and in the CDK2/CycA Complex are indicated, with CDK2 and Cyclin. The T-loop, ATP, PSTAIRE helix and helices involved in cyclin interactions with the CDK are shown.

FIG. 1B: Fluorescence spectrum of CDKCONF-Cy3. CDKCONF-Cy3 was engineered by mutation of Cys118Ser and fluorescent labelling of C177 and C199 with Cy3-maleimide.

FIG. 1C: Gel-electrophoresis analysis of expression of GST-Fusions of CDK2 and CDKCONF in E. coli (60 kDa).

FIG. 1D: Gel-electrophoresis analysis of the result of phosphorylation of 50 uM Histone H1 with gamma33-P-ATP with 100 nM CDK2 or CDKCONF complexed with cyclin A.

FIG. 2: In vitro characterization of CDKCONF

The graph represents CDKCONF relative fluorescence as a function of concentration of: ADP (dark circles), C4 peptide (clear circles) which inserts at the interface between CDK2 and cyclin A, the ATP-binding pocket inhibitor roscovitine (clear triangles) and CyclinA (dark squares). Data were corrected and fitted using Grafit software.

FIG. 3: Fluorescence imaging of cellular internalization of CDKCONF into living HS68 fibroblasts

500 nM CDKCONF labelled with FITC was introduced into HS68 fibroblasts using the PEP1 cell-penetrating peptide at a ratio CDKCONF/PEP1 of 1/15 (Morris et al. Nat. Biotechnol. 2001). Fluorescence imaging reveals that cells in G2 and entering mitosis are brighter than cells in other cell cycle stages.

FIGS. 4A and 4B: Characterization of the eight best compounds affecting CDKCONF fluorescence

FIG. 4A: Effect of the ten best compounds on proliferation of A549 cells. The graph (left side of FIG. 4A) shows, for each of the eight compounds, the percentage of A549 viability expressed as a function of the compound concentration. The table (right side of FIG. 4A) shows, for each compound, the calculated EC50.

FIG. 4B: Effect of the eight best compounds on CDK2/Cyclin A activity in vitro. For each of the compounds, the histogram shows the percentage of relative radioactivity incorporated into the histone H1 substrate by active CDK2/Cyclin A incubated with compounds at concentrations from 10 nM to 100 microM.

FIG. 5: Mechanism of action/interaction of the four best compounds with CDK2 and CyclinA

For each of the four compounds (from left to right, molecule 4 to 7), a graph shows the measured relative fluorescence of these molecules incubated with CDK2 (diagmonds), Cyclin A (white circles) or the CDK2/Cyclin A complex (black triangles), as a function of protein concentration in nM.

FIGS. 6A and 6B: Docking of the best compound onto CDK2/CyclinA

FIG. 6A: Representation of the docking of compound 4 on the CDK2/Cyclin A complex

FIG. 6B: Fluorescence titration of CDKCONF-Cy3 (double mutant Cys177 Cys191) and of single fluorophore mutants of CDKCONF: Cys177 and Cys191, with compound 4. The relative fluorescence is shown as a function of compound 4 concentration.

FIG. 7: Fluorescence titration of CDKCONF4

This graph represents the relative fluorescence of CDKCONF4, derived from CDK4, as a function of concentration (in nM) of: GST-cyclin D (black circles), GST-Retinoblastoma protein (squares) and MP-38 peptide which binds to the PISTVRE helix of CDK4 (triangles). Data were corrected and fitted using Grafit software.

FIG. 8: Identification of chemical compounds modifying CDKCONF4 conformation

This histogram represents the relative fluorescence of CDKCONF4 in the presence of 15 hit chemical compounds (named B5 to G9). On the left of the figure, the 4a column corresponds to CDKCONF4 alone (100%), “ctrl +” is a positive control in presence of Cyclin D, “ctrl −” is a negative control in presence of MP38 peptide, PD is a negative control in presence of the ATP analog PD-0332991.

### EXAMPLES

### Example 1

### Design and Production of CDKCONF, a Conformation-Sensitive Biosensor of CDK2

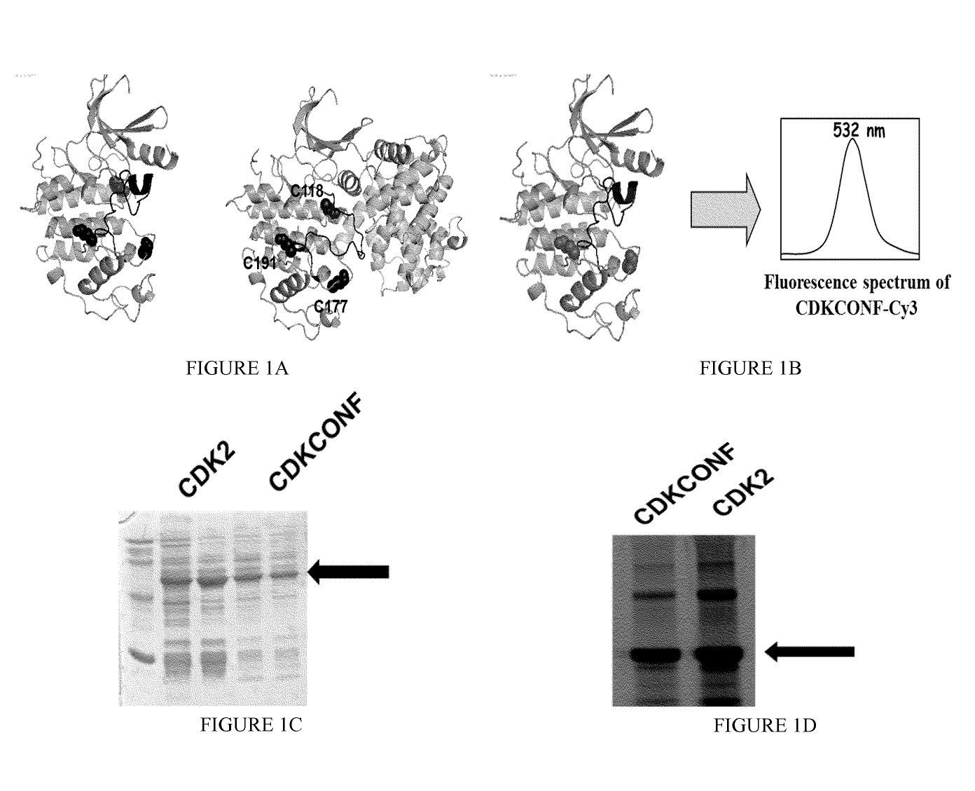

In order to generate a sensor that would report on changes that affect the conformation of the C-terminal lobe/T-loop of CDK2, it was chosen to exploit the cysteine residues present within the Cterminal lobe, and use them to couple synthetic fluorophores that are sensitive to changes in their environment. CDK2 bears three cysteine residues which lie within the C-terminal lobe C118, 177 and 191 (SEQ ID No 3), the two latter at positions which are particularly sensitive to conformational changes of the activation loop. C177 lies directly beneath T160, at the tip of the activation loop, and is therefore particularly well exposed for sensing molecules that bind and/or affect the conformation of the T-loop, and is also very close to Y179, which contacts the cyclin. C191 lies within alpha helix 3, that immediately follows the Tloop, and is at a position in close contact with W167 and Y168 (FIG. 1A). Preliminary experiments revealed that Cys177 and Cys191 serve as sensors of the T-loop dynamics when they are labelled with an-environmentally-sensitive dye, whereas Cys118 is not necessary. As such, a mutant of CDK2, termed CDKCONF (SEQ ID No 14) was engineered, in which cysteine 118 was mutagenized to serine, thereby preserving cysteine 177 and 191 for chemical coupling to thiol-labelling probes.

Other mutants of CDK2 were generated, wherein Cys118 and Cys117 were mutagenized to Serine (C118S/C177S) or wherein Cys118 and Cys191 were mutagenized to Serine (C118S/C191S), thereby producing respectively C177 CDK2 and C191 CK2. These mutants were produced and labeled as indicated for CDKCONF.

### CDKCONF Expression, Purification and Labelling

CDKCONF was expressed as a GST fusion in E. coli by IPTG induction 3 h at 37° C., as described in Gondeau et al. (JBC 2005) then purified by FPLC affinity and size exclusion chromatography on GST-Trap and Superdex75 columns, respectively, in TBS buffer. The purity of CDKCONF was then verified on SDS-PAGE and its concentration determined by measuring absorbance at 280 nm (FIG. 1C). CDKCONF was then labelled according to the manufacturer's instructions with tenfold molar excess Cy3 maleimide (GE Healthcare) or Fluorescein maleimide (Pierce) and further purified from free label on NAP-5 columns. The fluorescence spectrum of CDKCONF-Cy3 was checked (FIG. 1B).

We further verified whether CDKCONF was still as enzymatically active as wild type CDK2, following complexation with cyclinA and activation by Cdk Activating Kinase of S. cerevisiae (CIV) (FIG. 1D). CDKCONF is indeed as active as CDK2 towards histone H1 when complexed with cyclinA and incubated with CIV.

### Example 2

### Characterization of CDKCONF

CDKCONF was characterized in vitro to determine its sensitivity and specificity. We first performed a series of binding/titration experiments, with cofactor nucleotides, ATP-analogs inhibitors (Roscovitine), and interfacial peptide inhibitors of CDK2/Cyclin A (C4). Tests were performed as described in Gondeau et al, (2005).

### Steady-State Fluorescence Experiments

Fluorescence experiments were performed at 25° C., using a SPEX-PTI spectrofluorimeter in a 1 cm path-length quartz cuvette, with a band-pass of 2 nm for excitation and emission, respectively. Excitation was performed at 550 nm and emission spectra were recorded from 560 to 580 nm. A fixed concentration of Cy3-labelled CDKCONF (100 nM) was titrated with increasing concentrations of nucleotide, ATP analogs or peptide inhibitors from 25 nM to 1 μM. Data were fitted as previously described using a quadratic equation (GraFit, Erithacus Software).

### Results

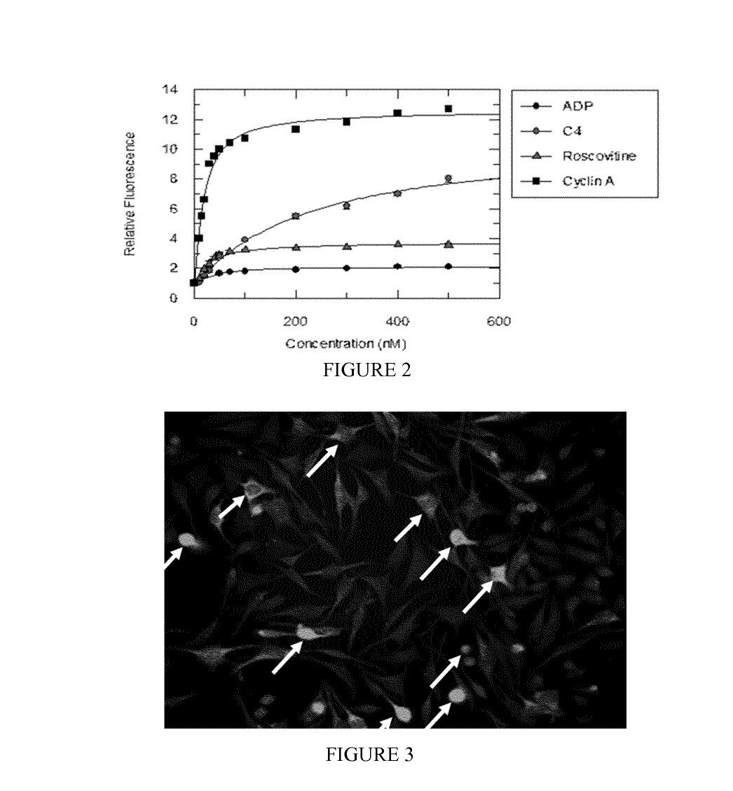

The fluorescence of CDKCONF, derived from CDK2, is significantly enhanced upon interaction with partners such as cyclins or molecules that interfere with the conformational dynamics of the T-loop, and which therefore affect CDK activation. On the contrary CDKCONF fluorescence is barely affected by ATP or ATP analogs or derived inhibitors that bind the ATP-binding pocket within the N-terminal lobe of the CDK. The sequence of this biosensor is original and its potential is directly related to its sensitivity of detection of molecules that affect the conformation of the T-loop. The sensitivity is directly related to the position at which the fluorescence probes are coupled (FIG. 2).

These experiments show that CDKCONF constitutes an excellent probe for identification of CDK inhibitors that affect the conformation of CDK2, not ATP-analogs that bind the N-terminal lobe, far from the T-loop. Titration of CDKCONF-Cy3 with either ATP or ADP, up to concentrations of 20 uM did not yield any significant changes in fluorescence of CDKCONF-Cy3.

In contrast, titration with a small peptide C4 that inserts between CDK2 and Cyclin A, not far from the T-loop (Gondeau et al., 2005) induced a significant increase in fluorescence of Cy3 upon excitation at 550 nm, with a shift in the emission maximum from 555 nm to 558 nm. Further, titration of CDKCONF-Cy3 with GST-cyclinA lead to a dramatic increase in fluorescence, indicative of a direct interaction with perturbs the Clobe, whereas GST did not have any effect (FIG. 2).

The experiments with individually labeled C177 CDK2 and C191 CDK2 show that they can serve as sensitive reporter of conformational changes in CDK2 upon interaction with CyclinA, although to a lesser extent than CDKCONF.

TABLE 2

| | | Maximum |
|---|---|---|
| | | Fluorescence |
| Labelling | Kd (nM) | Enhancement |
| C177 & | Cyclin A: 50 | 12× |
| C191-FITC | | |
| C191-FITC | Cyclin A: 45 | 5× |
| C177-FITC | Cyclin A: 50 | 7× |

### Example 3

### Application of CDKCONF to Monitor CDK2 Activation/Inhibition in Living Cells

### Cell Culture and Microscopy

HeLa cells were cultured in DMEM supplemented with 10% FCS, at 37° C. in an atmosphere containing 5% CO₂. Cell culture media, serum and antibiotics were purchased from Invitrogen, France. Epifluorescence images were acquired on a ZEISS AxioImager Z1 microscope with a Plan Apo 20× or 40× objective equipped with a Coolsnap HQ camera (Photometrics) and piloted by the Metamorph software (Universal Imaging).

In order to introduce the CDKCONF biosensor into living cells (Morris et al. 2001), we combined CDKCONF biosensor labelled with FITC with an efficient cell-penetrating peptide known as Pep1 at a CDKCONF:Pep1 molar ratio of 1:15. Association of this biosensor with a cell-penetrating peptide enables its direct application to cultured mammalian cells. Cell delivery was performed as described in Morris et al. (2001).

### Results

Observation of cells by fluorescence microscopy revealed that cells in G2 and undergoing mitosis, in which CDK2 is known to be associated with its cyclin partner and in an active conformation, CDKCONF-FITC fluorescence was significantly greater than in other cells (FIG. 3, cells with arrows show greater fluorescence of CDKCONF-FITC).

### Example 4

### Application of CDKCONF to the Identification of Chemical Modulators of CDK2 In Vitro

CDKCONF-Cy3 was applied to high throughput screen of a library of small molecule chemical compounds, to select inhibitors that would affect CDK/cyclin activation without targeting the ATP-binding pocket.

### Materials and Methods

Stability assays, sensitivity to DMSO, and downscaling experiments were undertaken to transfer CDKCONF-Cy3 to a miniaturized screen in 384-well plates. Assay conditions were miniaturized for 384-well plates and optimized so as to obtain a robust and reproducible signal. This assay was first validated with the C4 peptide (Gondeau et al., 2005), used as a positive control reference to measure an increase in CDKCONF-Cy3 fluorescence. The increase in fluorescence was calculated with reference to basal fluorescence of CDKCONF-Cy3 and is expressed as the percentage of increase. The assay buffer is 50 mM Tris-HCL ph 7.5, 150 mM NaCl, 1 mM EDTA and 10% glycerol. CDKCONF (5 nM) was stabilized in the assay buffer with 1% DMSO, during 1 hour at room temperature and protected from light. After 45 minutes of incubation, the Cy3 Fluorescence was quantified with an Envision® plate reader (Perkin Elmer, USA) using an excitation filter at 531 nm (BODIPY TMR531, Perkin Elmer) and an emission filter at 579 nm (Emission 579, Perkin Elmer). The C4 peptide, stored in stock solution 10-3M was tested in the assay at 5 μM. The molecules from the library are tested at 10-5M final.

Performance assays involving reproducibility, sensitivity, tolerance and robustness were undertaken with ATP and two ATP analogs, roscovitine and staurosporine, and the C4 peptide, as a positive control, since it promotes significant fluorescence enhancement.

### 1. Large Scale Determination of CDKCONF Fluorescence Modification

22.272 chemical compounds were obtained from a library of small chemical compounds. These compounds were tested at 10-5M. Hits were identified based on their ability to enhance fluorescence of CDKCONF-Cy3 at least as significantly as did the C4 peptide. A total of 264 compounds out of the 22.272 (1.18%) lead to a significant increase in fluorescence, as previously defined.

### 2. Elimination of Compounds Autofluorescent at Wavelength Used for the Assay

201 of these 264 compounds (0.9% of the library) are autofluorescent at the wavelength used in the assay (579 nm). Since intrinsic fluorescence of these compounds may mask their true activity, they were excluded from the rest of the study.

### 3. Manual Determination of CDKCONF Fluorescence Modification

The 63 remaining compounds (0.28% of the library) (liquid stocks in DMSO) were retested by individual manual confirmation. In order to obtain robust screening data, we applied a manual procedure based on the automated procedure to reconfirm the positive hits independently. The activity of the 63 original hits was reevaluated in quadruplate from a liquid stock of the compounds stored at −20° C. 47 out of the original 63 hits (73%, or 0.2% of the compound library) were retained. Among these confirmed hits, we marked those that lead to a signal amplification which was similar to or superior than amplification of the C4-A peptide (reference peptide).

### 4. Determination of IC50s

The 47 molecules from previous step were characterized in vitro to determine the dose-dependent modification of fluorescence by identified compounds on CDKCONF (IC50). The 47 positive hits obtained from the screen were characterized further in vitro, so as to determine their potency towards CDKCONF in a fluorescence assay, from solid stocks (powder). The percentage of amplification is expressed as a function of the concentration of the compound. The dose-dependent effect of the compounds on CDKCONF fluorescent was plotted thereby defining four different groups of compounds with different behaviour with respect to CDKCONF fluorescence.

From these 47 molecules, compounds were selected for their ability to raise the fluorescence signal by at least 50%, when compared to initial signal, for a concentration of compound comprised between 10-4M and 10-5M. Eight compounds were selected and further characterized to assess their effect in terms of inhibition of in vivo cell proliferation and of in vitro kinase activity.

### 5. Characterization of Inhibition of CDK2/Cyclin a Activity on Cell Cycle Progression and Cell Proliferation

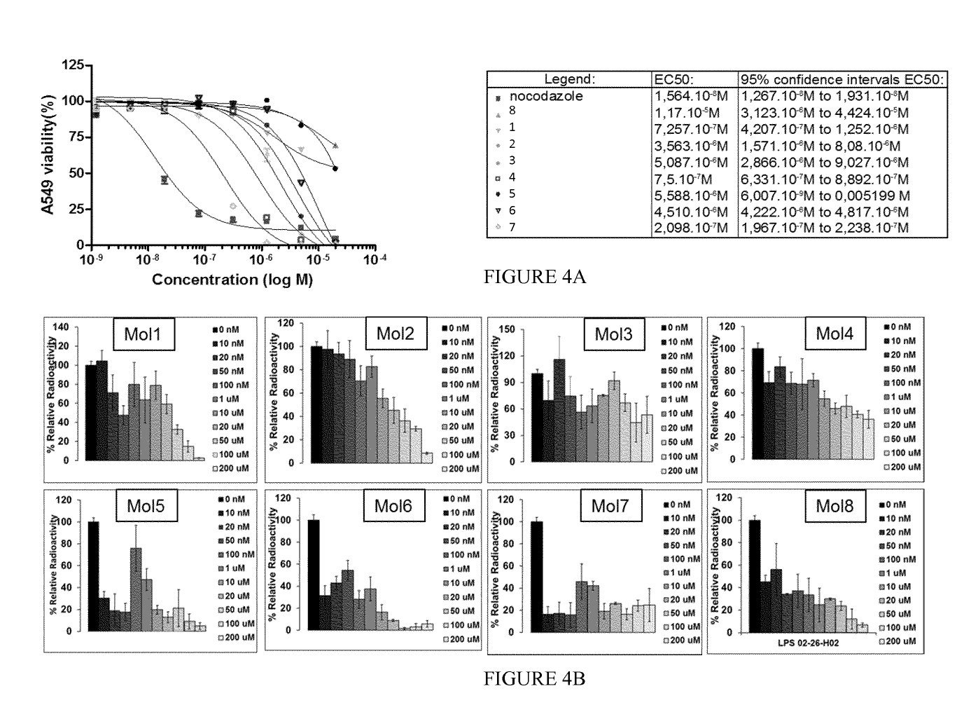

The inhibitory potential of these compounds on the proliferation of A549 cells, a liver tumor cell line, was determined in an MTT assay performed in triplicate and the EC50 values were determined from dose-dependent proliferation experiments. All eight molecules which were selected at step 4 had a significant effect on proliferation of A549 cells, with EC50 values ranging from 10-6 to 10-7M (FIG. 4A).

### 6. In Vitro Determination of the Inhibition of the Kinase Activity

The eight molecules from previous step were further assayed towards CDK2/Cyclin A kinase activity in a standard kinase assay using Histone H1 as a substrate, as described in Gondeau et al. JBC 2005. These experiments revealed net differences in the ability of molecules to inhibit kinase activity per se, with four molecules standing out with EC50 in the 20 nM range, whereas others only exhibited half inhibition of kinase activity at a concentration of about 20 uM (FIG. 4B).

### Conclusion

Based on these results, the four molecules, respectively compounds 4, 5, 6 and 7, presenting the greatest inhibitory potential towards CDK2/CycA both in the cell proliferation assay and in the in vitro kinase assay were retained as leads from this study.

### Example 5

### Mechanism of Action of Compounds 4, 5, 6 and 7, and Docking of Compound 4

To gain insight into the mechanism of action of these drugs, we first characterized their ability to interact with CDK2 and CyclinA. To this aim, we determined the intrinsic fluorescent properties of compounds 4, 5, 6 and 7.

| | Maximum | Maximum |
|---|---|---|
| | excitation (nm) | emission (nm) |
| Compound 4 | 324 | 422 |
| Compound 5 | 324 | 422 |
| Compound 6 | 326 | 420 |
| Compound 7 | 326 | 420 |

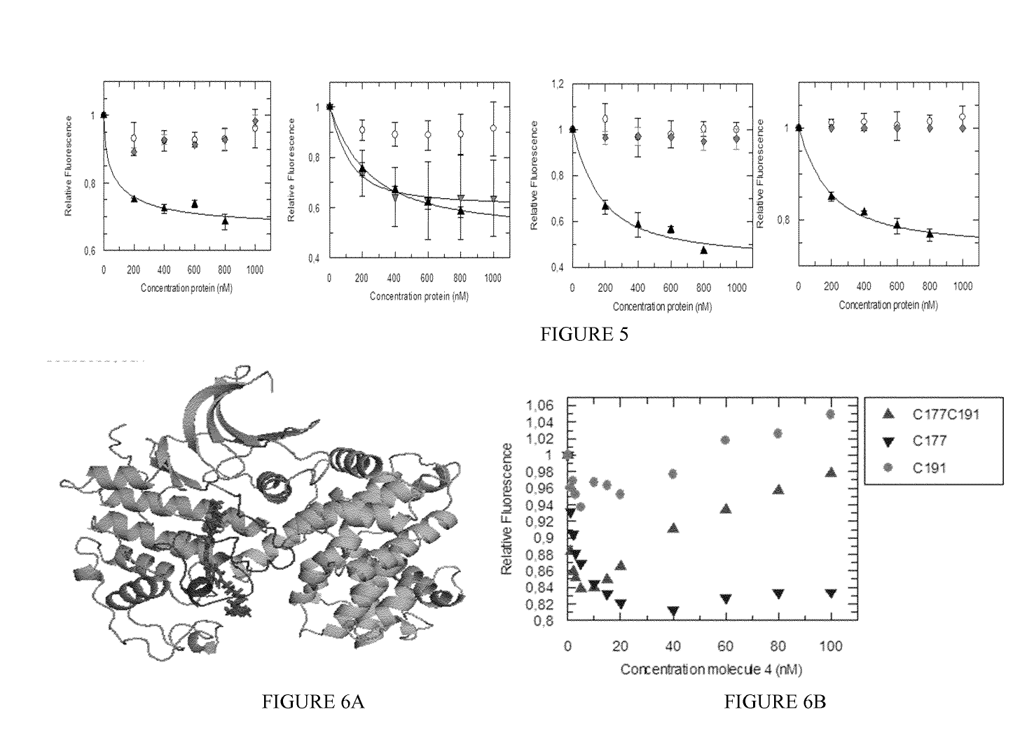

We took advantage of the intrinsic fluorescent properties of these molecules and performed fluorescence titration experiments in which changes in the fluorescence of these four molecules were monitored upon incubation with CDK2, CyclinA or the CDK2/CyclinA complex (FIG. 5). Characterization of compounds binding to CDK2 and CDK2/CyclinA complexes was monitored by measuring fluorescence at 460 nm following excitation at 355 nm on a Polarstar 96-well plate reader.

All four molecules underwent fluorescence quenching upon addition of increasing concentration of the GST-CDK2/Cyclin A complex. For compounds 4, 6 and 7, barely any change in fluorescence upon incubation with monomeric GST-CDK2 or CyclinA was noticed. On the contrary, for compound 5, upon incubation with monomeric GST-CDK2, a change in fluorescence was observed. This indicates that these molecules probably dock into a very different environment within the CDK2/cyclin complex than onto the CDK subunit alone.

Finally, docking studies were performed with the aim of identifying the potential binding site of the molecules onto CDKCONF, FIG. 6A. The relative fluorescence of CDKCONF or of single mutants Cys177 and Cys191 was determined in the presence of molecule 4. The results indicate that the binding site of molecule 4 is located near Cys177.

Dissociation constants were determined for the different compounds, they are the following:

TABLE 3

| | Kd (nM) |
|---|---|
| Complex vs Molecule 4 | 28 ± 26 |
| Complex vs Molecule 5 | 174 ± 26 |
| GST-Cdk2 vs Molecule 5 | 58 ± 22 |
| Complex vs Molecule 6 | 114 ± 64 |
| Complex vs Molecule 7 | 125 ± 35 |

### Example 6

### Design and Production of CDKCONF4, a Conformation-Sensitive Biosensor of CDK4

CDK4 bears four cysteine residues: Cys78, Cys135, Cys202 and Cys215, whereas residue 189 is a serine (SEQ ID No 5). A mutant of CDK4, termed CDKCONF4 was engineered, in which Cys78, Cys135 and Cys215 were mutagenized to Ser78, Ser135 and Ser215, respectively, and in which Ser189 was mutagenized to Cys189 (SEQ ID No 17). Therefore, CDKCONF4 comprises two Cysteine residues, Cys189 and Cys202, for chemical coupling to thiol-labelling probes.

### CDKCONF4 Expression, Purification and Labelling

CDKCONF4 was expressed as a GST fusion in E. coli by IPTG induction, then purified and labelled as described in Example 1 for CDKCONF.

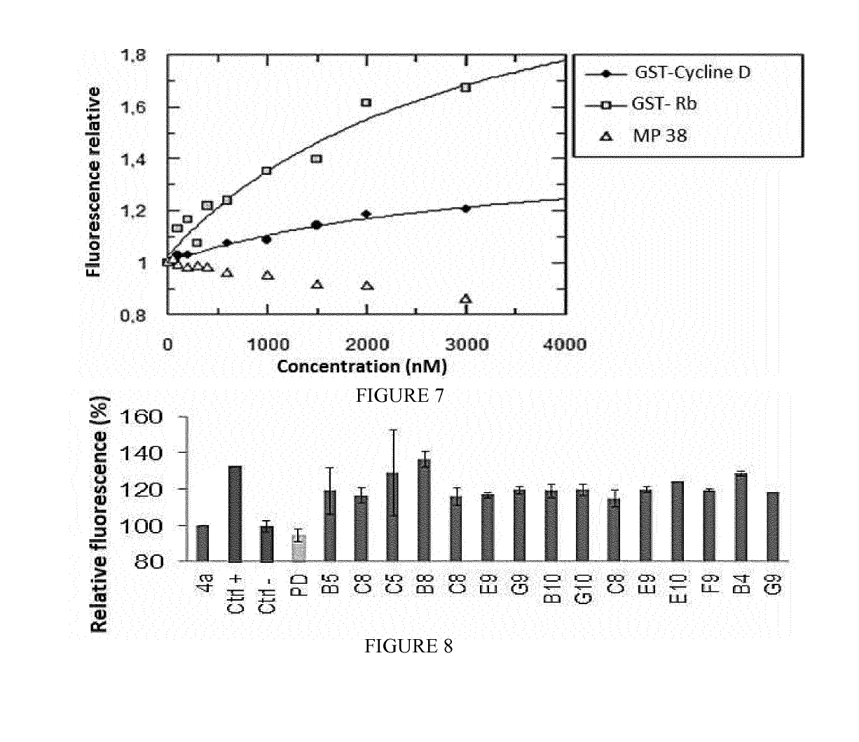

CDKCONF4 was then characterized in vitro to determine its sensitivity and specificity. A series of binding/titration experiments was performed in the presence of GST-cyclin D, GST-retinoblastoma protein or MP38 peptide (FIG. 7). Tests were performed as described in Gondeau et al., (2005) and in example 2.

### Results

The fluorescence of CDKCONF4, derived from CDK4, is significantly enhanced upon interaction with the Retinoblastoma protein, one of the major substrates of CDK4/Cyclin D. The fluorescence of CDKCONF4 is only very slightly affected upon interaction with Cyclin D, consistent with the lack of structural changes induced (Takaki et al., 2009; Day et al., 2009)

These experiments show that CDKCONF4 constitutes an excellent probe for identification of CDK inhibitors that induce conformational changes in CDK4.

### Example 7

### Application of CDKCONF4 to the Identification of Chemical Modulators of CDK4 In Vitro

CDKCONF4-Cy3 was applied to high throughput screen of a library of small molecule chemical compounds, to select inhibitors that would affect CDK/cyclin activation without targeting the ATP-binding pocket. The screen was performed using 25 nM CDKCONF-Cy3 in 200 ul phosphate saline buffer, 500 mM NaCl, pH 6.4. Excitation was performed at 550 nm and Emission acquired at 570 nm after 5 h incubation of CDKCONF4-Cy3 alone, or with positive control (Retinoblastoma at 2 uM) or negative controls (MP38 peptide or PD-0332991, or with the chemical compound. The Z factor of the screen was 0.68.

480 chemical compounds were obtained from the essential French National Library of small chemical compounds. These compounds were tested at 10-5 M. Hits were identified based on their ability to enhance fluorescence of CDKCONF4-Cy3 (FIG. 8). None of these compounds exhibited autofluorescence at 570 nm following excitation at 550 nm.

### BIBLIOGRAPHIC REFERENCES

1. Malumbres et al., Trends Biochem Sci., 30:630-641, 2005
2. Obaya et al., Cell Mol Life Sci., 59:126-142, 2002
3. Satyanarayana et al. Oncogene. 28:2925-2939, 2009
4. Merrick et al., Cell Cycle. 9:706-714, 2010
5. Jeffrey et al., Nature, 376(6538):313-20, 1995
6. Morgan et al., Annu Rev Cell Dev Biol., 13:261-291, 1997
7. Morris et al. J. Biol. Chem. June 28, 277(26):23847-53, 2002
8. Lolli et al., Cell Cycle., 9:1551-1561, 2010
9. Malumbres et al., Rev Cancer., 1:222-231, 2001
10. Malumbres at al., Curr Opin Genetics and Development., 17:60-65, 2007
11. Lapenna et al., Nat Rev Drug Discovery, 8:547-566, 2009
12. Malumbres et al., Nat Rev Cancer., 9:153-166, 2009
13. Harwell et al., J Biol Chem., 279:12695-12705, 2004
14. Ekberg et al., Eur J Haematol., 75:106-115, 2005
15. Husdal et al., Cell Oncol., 18:107-116, 2006
16. Suzuki et al., Cancer Sci., 98:644-651, 2007
17. Kim et al., J Cell Physiol. 220:292-296, 2009
18. Malumbres et al. Nat. Cell. Biol. 11:11, 1275-1276, 2009
19. Lolli G. et al., Cell Cycle, 9:8, 1151-1561, 2010
20. De Bondt H L et al. Nature, June 17, 363(6430):595-602, 1993
21. Jeffrey P D et al. Nature, July 27, 376(6538):313-20, 1995
22. Russo A A et al. Nat. Struct. Biol., August 3(8):696-700, 1996
23. Brown N R et al. Nat. Cell. Biol., November, 1(7):438-43, 1999
24. Higgins et al., “Protein Expression: A Practical Approach”, Oxford, UK: Oxford University Press
25. Baneyx F et al., “Protein Expression Technologies: Current Status and Future Trends”, Norfolk, UK: Horizon Bioscience
26. Atherton et al., “Solid Phase peptide synthesis: a practical approach”, Oxford, England.
27. Loving et al., Trends Biotechnol., 28(2):73-83, 2010.
28. Kurzawa et al, Biochim. Biophys. Acta, 210 December 1798(12), 2274-85, 2010
29. Morris et al., Nature Biotech., 19, 1173-1176, 2001
30. Gondeau et al., J Biol Chem. 2005 Apr. 8; 280(14):13793-800.
31. Grdisa et al., Curr. Med. Chem., 18(9), 1373-1379, 2011
32. Fonseca et al., Advanced Drug Delivery Reviews, 61, 953-964, 2009
33. Morris et al., Biol. Cell., 100, 201-217, 2008
34. Takaki T et al., Proc Natl Acad Sci USA. 2009 Mar. 17; 106(11):4171-6.
35. Day P J et al., Proc Natl Acad Sci USA. 2009 Mar. 17; 106(11):4166-70.

1. A compound comprising a polypeptide and at least one fluorophore, wherein:
a) the amino acid sequence of said polypeptide comprises the sequence V/IVTXWYRXPXI/V/LL (SEQ ID NO:1) and is derived from the amino acid sequence of at least one sequence of a protein from the CDK family, and
b) said at least one fluorophore is coupled to an amino acid of said polypeptide, wherein the position of said amino acid within the sequence of said polypeptide is chosen from the group consisting of: position 6 to position 36, wherein position 1 within the sequence of said polypeptide is defined as the position of the first amino acid of said sequence V/IVTXWYRXPXI/V/LL (SEQ ID NO:1).

2. A compound according to claim 1, wherein said polypeptide comprises an amino acid sequence derived from at least one sequence from the list consisting of:
a) the human proteins CDK1 (SEQ ID NO:2), CDK2 (SEQ ID NO:3), CDK3 (SEQ ID NO:4), CDK4 (SEQ ID NO:5), CDK5 (SEQ ID NO:6), CDK6 (SEQ ID NO:7), CDK7 (SEQ ID NO:8), CDK8 (SEQ ID NO:9), CDK9 (SEQ ID NO:10), and CDK10 (SEQ ID NO:11),
b) the Saccharomyces cerevisiae protein CDC28 (SEQ ID NO:12), and c) the Schizosaccharomyces pombe protein CDC2 (SEQ ID NO:13).

3. A compound according to claim 1, wherein the sequence of said polypeptide comprises at most two amino acid residues comprising a thiol group, wherein the position within the sequence of said polypeptide of said at most two amino acid residue comprising a thiol group is chosen from the group consisting of: position 6 to position 36.

4. A compound according to claim 1, wherein the sequence of said polypeptide is chosen in the list consisting of: SEQ ID NO:14, SEQ ID NO:15, SEQ ID NO:16, SEQ ID NO:17, SEQ ID NO:18, SEQ ID NO:19, SEQ ID NO:20, SEQ ID NO:21, and SEQ ID NO:22.

5. A compound according to claim 1, wherein the compound comprises two fluorophores, with each fluorophore being coupled to an amino acid residue of said polypeptide.

6. A compound according to claim 1, wherein said at least one fluorophore is coupled to a cysteine residue.

7. A compound according to claim 1, wherein said at least one fluorophore is an environmentally sensitive dye.

8. A compound according to claim 1, wherein said at least one fluorophore is Cy3.

9. A compound according to claim 1, wherein the polypeptide sequence has the sequence chosen in the list consisting of: SEQ ID NO:14, SEQ ID NO:15, SEQ ID NO:16, SEQ ID NO:17, SEQ ID NO:18, SEQ ID NO:19, SEQ ID NO:20, SEQ ID NO:21, and SEQ ID NO:22 and wherein two fluorophores are coupled to the polypeptide on cysteine residues and are Cy3 fluorophores.

10. A compound according to claim 1, characterized in that it comprises a protein tag or a cell-penetrating peptide sequence

11. A compound according to claim 1, used as a conformation-sensitive biosensor.

12. A method for determining if a product is capable of modulating the conformation of at least one CDK, said method comprising the steps of:
a) providing at least one compound according claim 1,
b) contacting said compound with said product,
c) illuminating said compound and product with an excitation light,
d) determining the fluorescent signal emitted by said compound,
e) comparing said fluorescent signal with a reference fluorescent signal, and
f) determining from the comparison of step e) if said product is capable of modulating the conformation of at least one CDK.

13. The method according to claim 12, which is a method for screening a plurality of products for their ability to modulate the conformation of at least one CDK, said method comprising the following steps:
a) providing at least one compound according to claim 1,
b) contacting said compound with at least one of said products,
c) illuminating said compound and said at least one product with an excitation light,
d) determining the fluorescent signal emitted by said compound,
e) comparing said fluorescent signal with a reference fluorescent signal, and
f) determining, from the comparison of step e), the ability of said product to modulate the conformation of at least one CDK.

14. A kit comprising at least one compound according to claim 1 and an acceptable carrier and/or an acceptable solvent.

15. The use of a compound according to claim 1 for the screening of a plurality of products for their ability of modulating the conformation of at least one CDK.
